(19) United States
(12) Patent Application Publication
(10) Pub. No.: US 2015/0240716 A1
(43) Pub. Date: Aug. 27, 2015

(54) Ecological Sequestration of Carbon Dioxide/Increase of Bio-Energy Obtainable Through Biomass

(71) Applicant: Karl Werner Dietrich, Odenthal (DE)

(72) Inventor: Karl Werner Dietrich, Odenthal (DE)

(21) Appl. No.: 14/426,002

(22) PCT Filed: Sep. 4, 2012
(86) PCT No.: PCT/DE2012/000883

§ 371 Date: Mar. 18, 2015

(51) Int. Cl. F02C 3/22; C10L 3/10; C25B 1/04; C07C 1/12

(52) U.S. Cl. CPC F02C 3/22; C07C 1/12; C10L 3/10; C25B 1/04

(57) ABSTRACT

According to known methods, biomass is broken down under the action of water vapour via a carbon monoxide-hydrogen mixture (called synthesis gas) as an intermediate stage into hydrogen and carbon dioxide instead of being combusted directly to generate energy. Carbon dioxide is stored/sequestered and the hydrogen is used to generate energy. The transfer of bio-activity can also be effected within the same process by breaking down a mixture of biomass and fossil fuel (e.g. wood and coal) into carbon dioxide and hydrogen. The hydrogen is then reacted with half of the formed carbon dioxide to form methane and the remaining carbon dioxide is stored. The stored carbon dioxide and generated methane respectively comprise one half each of biological and fossil carbon. If the bio-activity of the stored bio-carbon dioxide is transferred to the fossil carbon in methane, a corresponding mixture of wood and coal produces 100% biomethane. Here, too, up to 100% biomethane can be obtained from coal-wood mixtures. By adding the hydrogen obtained from excess electrical energy to the biocarbon, the bio-energy based on the biomass used is even quadrupled. For a traceable eco-balance with such mixtures, it is important to quantify the bio-proportion in the two “end products” stored carbon dioxide and generated methane. For this purpose, use is made e.g. of the radiocarbon (C14) method.

The sequestration of carbon dioxide (CO₂), also referred to as CCS (carbon capturing and storage), is usually employed for CO₂ from the combustion of fossil carbon compounds not to be released into the atmosphere. In this process, the CO₂ is pressed into underground cavities under a high pressure. Exhausted natural gas fields are often employed as cavities. Because it is not established with certainty that the CO₂ will remain enclosed underground permanently, the acceptance of CCS is low for the time being. Nevertheless, CCS will not be avoidable if the society further depends on power generation from fossil fuels, and the latter must be combusted in a CO₂-neutral way (without CO₂ emission).

Another way of CO₂-neutral power generation is the combustion of biomass or of reaction products from biomass, such as biogas, bioalcohol, or biodiesel. In this case, it is considered that the CO₂ released during the combustion was previously taken up by the plant during photosynthesis and thus withdrawn from the atmosphere.

Of course, power can also be generated without emissions by means of wind and the sun. It is even possible to convert wind or solar power to hydrogen by electrolyzing water, and to combust it without emissions. Hydrogen burns exclusively to water vapor.

Evidently, there are enough methods to generate power without emissions. Meanwhile, fossil carbon is still being burnt, and the society is hesitatingly willing to at least restrict the emission of CO₂. As yet, there is no feasible method for recovering the CO₂ from the atmosphere.

Such a method would exist if biomass was decomposed into 2 moles of hydrogen and 1 mole of CO₂, the two gases were separated, the CO₂ was sequestered, and the hydrogen was burnt for power generation. As mentioned above, hydrogen burns to water vapor without emissions. The CO₂ that was withdrawn from the atmosphere by the employed biomass in the growing season is permanently stored underground, and the biomass is nevertheless burnt without emissions. In the balance, energy generation is combined with the sequestering of carbon dioxide from the atmosphere.

Thus, an essential part of the present invention is the ecological sequestering of carbon dioxide, characterized in that biomass is thermally or chemically converted to carbon dioxide and hydrogen using steam, carbon dioxide and hydrogen are separated, then carbon dioxide is stored/sequestered to generate a “climate credit”, and the hydrogen is used for power generation.

The biomass includes all biological agricultural and forestry raw materials containing carbon and hydrogen. As examples of raw materials, there may be mentioned: wheat, corn, grass and wood, and agricultural and forestry waste. Of course, synthetic organic compounds may also be reacted with the biomass to form hydrogen.

The reaction products of biomass include all reaction products of biomass, such as biogas, bioalcohol or biodiesel, as well as fats, oils, sugars, cellulose, waxes.

The conversion of biomass or its reaction products to CO₂ and H₂ is preferably effected under pressure and heat with steam in a so-called reformer.

In the exhausted natural gas deposits, into which the CO₂ is to be pressed, the storage capacity is limited if natural gas is still present therein. For as a rule, a considerable proportion (up to 40%) of non recoverable natural gas remains in gas fields.

Now, because of its extremely low density (⅛ of that of natural gas) and its high flowability, the hydrogen obtained from the biomass can replace natural gas that is still enclosed, and drive it out of the deposit. Additional natural gas is extracted thereby, and at the same time, additional storage space for CO₂ becomes available. It can also be assumed that in the pores where the previously retained natural gas was first displaced by hydrogen and then replaced by CO₂, the CO₂ is absorbed by the rock and is therefore stored at low pressure.

Thus, the present invention further relates to the thermal and chemical conversion of biomass or its reaction products to carbon dioxide and hydrogen, characterized in that only the hydrogen is at first introduced into a natural gas deposit to extract/displace the natural gas from the deposit, then the CO₂ is sequestered, and the hydrogen is extracted/displaced by the introduced CO₂.

In order to avoid mixing during the extraction/displacement of the individual gases as far as possible, the large difference in densities between hydrogen on the one hand and methane (natural gas) and CO₂ on the other is utilized (hydrogen/natural gas=⅛, hydrogen/CO₂= 1/22). The procedure is such that hydrogen, which is light-weight, is introduced into the upper part of the deposit at first, and the natural gas to be extracted is withdrawn from the lower part. In the following sequestering of CO₂, the CO₂, which is heavy, is introduced into the lower part of the deposit, and the hydrogen is withdrawn from the upper part. From the time when a mixture of gases is extracted, the gases can be separated, and the respective kind of gas that is being introduced is recirculated back into the deposit.

If a hydrogen/natural gas mixture occurs on the extraction side during the introducing of hydrogen, one can either separate the hydrogen and recirculate it into the deposit as described above, or the mixture is passed through the network or through a particular line to the points of use. Since naturally the gases in the deposit will not mix uniformly, a fluctuating gas mixture is extracted. Because of the great physical and combustion-technological differences between hydrogen and natural gas, especially because of the different calorific values (the calorific value of natural gas is about three times higher than that of hydrogen), the current hydrogen content must be determined at the point of use, and the metering of the gas to the burner must be adjusted accordingly. Also the counter measuring the consumed energy must consider the hydrogen content. Because the necessary equipment is difficult to realize in private households, it is recommendable to feed the natural gas or the mixture of natural gas/hydrogen to large points of use in this concept of extraction/displacement, where the corresponding measuring devices can be provided. As examples, there may be mentioned heating plants or gas power plants.

Because of the fact that the calorific value of natural gas is three times higher than that of hydrogen, it is worth to continue the extraction even with low proportions of natural gas in the gas mixture. For example, only 20% natural gas content in a mixture of natural gas/hydrogen contributes to the calorific value of the mixture by almost 50%.

However, it is also possible to prepare a uniform mixture of hydrogen/natural gas after the extraction by adding hydrogen or natural gas to the fluctuating gas mixture according to need.

If the process according to the invention is transferred to coal seams or mines, it is also possible, as described, to first extract/displace the methane (mine gas) with hydrogen, and then to replace the hydrogen by the CO₂ to be stored. Mine gas usually also contains incombustible gases, which can make combustion inefficient. In such a case, it may be advantageous to increase the hydrogen content deliberately and thus to improve the energy density of the gas mixture.

In addition to the ecological importance, this process is also characterized by its high economic efficiency. This can be shown by the following calculation: From methane, hydrogen is obtained in four times the gas quantity in a steam reformer. Accordingly, from biogas with 50% to 80% methane, hydrogen can be obtained in twice to three times the gas volume. The natural gas extracted/displaced with this hydrogen then has three times the calorific value as compared to hydrogen. This calculation is illustrative and can be transferred to other classes of chemical compounds of the biomass.

Further, it has to be considered that natural gas that cannot be extracted conventionally is being extracted here. This fact alone causes an increase of the value of the biomass employed to three times its value. Into the bargain, as an energy source, there is the hydrogen that displaced the natural gas and is available when it is itself extracted/displaced by the subsequent CO₂.

The hydrogen that displaces the natural gas is a renewable resource by definition. Consequently, the part of the extracted natural gas that is equivalent to the originally employed biomass also becomes a renewable resource and can be burnt is gas power plants for power generation in a CO₂-neutral way. This is justifiable because the hydrogen issued from the biomass burns to water vapor without emissions, as mentioned above. The emission rights involved in the sequestering can then be transferred to another power plant. In other words, the emission rights or the bioactivity of the bio-carbon dioxide extracted from the atmosphere and permanently stored in the soil is transferred to fuels with fossil carbon.

Thus, the present invention relates to a process for increasing the bioenergy achievable with biomass, characterized in that biomass is converted chemically, for example, using steam, or thermally to carbon dioxide and hydrogen, the carbon dioxide is stored/sequestered, and the hydrogen is used for power generation, further in that emission rights involved in the sequestering of the biological carbon dioxide derived from the combustion/conversion of biomass to electricity or chemical separation from biomass are transferred to the emission of carbon dioxide produced in the combustion of fossil carbon (or that the bioactivity of the stored bio-carbon dioxide is transferred to the carbon of a fossil fuel). Thus, the biological carbon dioxide must be determined quantitatively and transferred proportionally to fossil carbon (natural gas).

The process according to the invention allows a continuous transition from the energy source natural gas to hydrogen (or through methane obtained therefrom, see below) as an energy source of renewable energies. For this process, no new lines, no additional power plants and no further stores are required. The process supplements the fluctuating wind and solar energies and together with them creates the ideal energy mix for the energy transition.

For a better handling as an energy source, hydrogen may also be converted to methane (methanized). This methane can then be supplied to the natural gas network and transported and consumed together with the natural gas. Natural gas predominantly consists of methane. As a reaction partner of the hydrogen, the sequestered/stored carbon dioxide suggests itself. In this case, 4 moles of hydrogen is required according to the following reaction (Reaction 1). This means, half of the carbon dioxide to be stored is needed for the methanization of the hydrogen obtained according to the invention. The other half is sequestered/stored.
Reaction 1 CO₂+4H₂→CH₄+2H₂O (Reaction 6 in the Annex)

The hydrogen may also be reacted with the so-called synthesis gas (mixture of carbon monoxide and hydrogen) obtained as an intermediate product in the reaction of carbon with water (Reaction 2). In this case, only 2 moles of hydrogen is additionally required for the production of methane:

Reaction 2 (CO+H₂)+2H₂→CH₄+H₂O

For this purpose, the synthesis gas is divided in the intermediate stage. One part thereof (about half) is “reaction to completion” to form CO₂ and hydrogen, and the remainder is reacted with the total formed hydrogen to methane according to Reaction 2. This means, for half of the synthesis gas, the additional process step of reacting the carbon monoxide to carbon dioxide is saved.

In the last paragraph of the chapter “Description of the Starting Materials” (Chapter 1 in the Annex), reference is made to the economical importance of the inclusion of fossil fuels, for example, coal, in the process according to the invention. Thus, for example, a mixture of wood and coal in which the ratio of fossil to biological carbon is 1:1 is converted to synthesis gas in the manner described above, the synthesis gas is divided, one part is converted to carbon dioxide and hydrogen, the carbon dioxide is stored, and the hydrogen is reacted with the remaining part of the synthesis gas to methane according to Reaction 2.

Then, the stored/sequestered carbon dioxide is based half on biological carbon, and half on fossil carbon. The same applies to the carbon of the methane. Now, if the bioactivity of the biological carbon in the stored carbon dioxide is transferred to the fossil carbon in the methane, 100% biomethane is obtained in the process according to the invention. In the life cycle assessment, the “climate bonus” (the bioactivity) of the biological carbon in the stored carbon dioxide is credited to the fossil carbon in the produced methane (cf. Chapter “Chemical Compounds/Mass/Volume/Energy”).

The synthesis gas may also be reacted with hydrogen additionally obtained from excess electric power by the electrolysis of water. Such hydrogen can be produced by the electrolysis of water, for example, from wind or solar power. This increases the proportion of synthesis gas that is reacted to methane. Then, the whole synthesis gas may also be converted to methane with hydrogen. By adding the excess electric power by means of the hydrogen to the original biocarbon obtained by transferring the bioactivity, the bioenergy is further multiplied in the thus produced biomethane.

According to this process, a storage power plant can be realized in which, in different operational stages, either carbon is converted to methane as described above, or further methane (practically double the amount) is produced with excess electric power by the electrolysis of water. However, the hydrogen produced from excess electric power may also be reacted with stored carbon dioxide.

The thus produced methane can be fed into the natural gas network, and in this way, fluctuating wind or solar power can be steadied, transported and reconverted to electric power from the stored and transported methane or its natural gas equivalents in a different place.

Another possibility to employ biomass or coal or mixtures of coal and biomass in an economically operating storage power plant for excess electric power is, in two successive operational stages:
1. to convert the synthesis gas to electric power in a gas power plant and thereby close supply gaps, and to store the formed carbon dioxide in the soil; or
2. to react the synthesis gas with hydrogen prepared from excess electric power by the electrolysis of water into methane, and to store the methane in the gas network.

In the first operational stage, electric power is supplied to the power grid. In the second operational stage, (excess) electric power is withdrawn from the power grid, converted to methane, and the methane is supplied to and stored in the gas network. The plant can be employed to stabilize the power grid, and as an energy storage facility. In the first operational stage, the bio-carbon dioxide whose bioactivity is transferred to methane formed with fossil carbon in the second operational stage is stored.

In such a storage power plant, an important synergy effect occurs: The connection of the power plant to the high voltage grid and to the transformers can be utilized in different directions, both to supply the energy in the first operational stage, and to withdraw electric power for the electrolysis of water in the second operational stage.

Such storage power plants are increasingly gaining importance, because with the further spread of fluctuating renewable energies in the near future, usually either too much or too little energy is contained in the electric power grid. The first operational stage then serves for covering supply gaps and peaks in demand, and the second operational stage serves for the storage and distribution of excess energy.

The conversion to electric power of the synthesis gas can be effected by:
burning the synthesis gas directly in the gas power plant. The combustion produces water vapor and carbon dioxide. After condensation of the water vapor, the carbon dioxide gas can be separated and sequestered from the remaining atmospheric nitrogen, for example, by liquefaction under pressure.
converting the carbon monoxide in the synthesis gas with steam to further hydrogen and carbon dioxide as described above, sequestering the carbon dioxide, and burning/converting to electric power the hydrogen.
converting part (ideally half) of the synthesis gas to hydrogen and carbon dioxide as above, sequestering the carbon dioxide, and converting the remaining part of the synthesis gas to methane by the hydrogen produced, and burning/converting to electric power the methane.

Because of the simpler procedure, the direct conversion of the synthesis gas to electric power in the power plant during the first operational stage of the storage power plant is preferred. If methane/natural gas is also converted to electric power in order to increase the power of the gas power plant, additional carbon dioxide to be stored is produced. The same applies to the water vapor formed in the combustion of methane, which is also condensed (see below).

Excess electric power is also obtained in all inflexible large power stations, such as coal and nuclear power plants, when the power grid cannot take up power from the power plant because of too high capacity utilization. This is a state that saps the economic efficiency of large power plants, and that becomes even more critical with the further spreading of renewable energies, because renewable energies have priority in the power grid.

In the conversion to electric energy of the synthesis gas in the first operational stage, the oxygen formed and stored in the electrolysis of water in the second operational stage can be used instead of the combustion air. This has the advantage that no nitrogen oxides, which are harmful to the climate, are formed in the absence of atmospheric nitrogen. This applies to all three kinds described of converting the synthesis gas to electric power.

Both in the combustion of the synthesis gas and in the combustion of methane with pure oxygen, carbon dioxide need no longer be separated from the flue gases. After condensation of the water vapor, carbon dioxide remains as the only gas and can be directly sequestered. Carbon monoxide, which is unavoidable in coal combustion, will then remain as a gas after liquefaction under pressure and can be recirculated into the burner, so that it is not released into the environment.

It is to be noted that in a combustion with pure oxygen, materials (e.g., of the gas turbine) arrive at the limit of thermal load capacity because of the high energy density of the combustion gas mixture. It is then recommendable to employ inert gases for “cooling”. Existing carbon dioxide or water vapor suggest themselves for this purpose.

Both in the combustion of hydrogen and in the combustion of the synthesis gas, water vapor occurs in the flue gases, which is substantially salt-free and similar to distilled water after condensation, and can be processed into feeding water for the electrolysis of water. The same applies to the combustion of methane. An aqueous condensate is also formed in the hydrogenation of carbon dioxide and carbon monoxide (Reaction 1 and Reaction 2). The condensed water often still contains traces of acid, which can be removed by anion exchangers.

The demand for highly purified feeding water for the electrolysis of water on the scale as provided here is enormous. Assuming that 1 million kW of excess energy is to be bound as hydrogen in the second operational stage by such a storage power plant, it would be necessary to provide 250,000 liters of distilled water. It is evident that the availability of purified water for the electrolysis of water is of critical importance to the economic efficiency of the overall process. With the different condensates from the combustion of hydrogen as listed above, such availability is achieved. Since the water is formed/burnt in the respective other operational stage, it must be stored in the water tank. The condensates mostly contain traces of acid, which are removed with anion exchangers.

The present technology enables carbon dioxide to be withdrawn from the atmosphere through the photosynthesis of plants (biomass), and after thermal utilization (combustion) of the biocarbon from these plants, to store/sequester the formed bio-carbon dioxide in the soil. Now, it becomes possible to burn fossil fuels, such as coal, at the same carbon ratio in a carbon dioxide-neutral way in a unitary process together with the biomass (e.g., wood). This means, the bio-carbon dioxide formed and sequestered in the first operational stage by combustion/conversion to electricity of the synthesis gas can provide an equimolar amount of fossil carbon with carbon dioxide-neutrality in combustion in the second operational stage.

The “climate leverage” according to the invention in the storage of bio-carbon dioxide can be utilized already in the conversion to electricity of the synthesis gas from corresponding mixtures of coal and wood. The effect is increased if additional hydrogen from excess electric power is obtained by the electrolysis of water in the energy storage stage (2nd operational stage), and the synthesis gas methanizes this hydrogen. The thus obtained methane can be converted to electricity on-site, or fed into the gas network, and can thus transport wind and solar power through gas lines. The multiplication of the bioenergy is effected inside the storage power plant.

The extent to which this technology combines economic efficiency with ecology becomes clear from the following Example: A composition of 120 tons of wood and 80 tons of coal (carbon content/calorific value: wood: 50%/4-5 kWh, coal: 75%/7 kWh) is converted to synthesis gas by gasification. Half of the synthesis gas is converted to electricity in the first operational stage, wherein about 300,000 kW of electric power is obtained (efficiency of coal gas conversion to electricity: about 50%). The carbon dioxide produced, which contains equal shares of biological and fossil carbon, is stored/sequestered. The second half of the synthesis gas is hydrogenated to methane with hydrogen obtained from 1 million kW of excess wind power in the second operational stage (Reaction 2) to obtain about 130,000 cubic meters of methane, which is itself composed of equal shares of biological and fossil carbon. This biomethane (see above), which is reconverted to electricity on-site or on a different site, yields about 850,000 kW of ecological electric power in a gas and steam power plant (efficiency: 60%). The efficiency based on the excess energy employed is 85% (cf. Chapter “Chemical Compounds/Mass/Volume/Energy” in the Annex).

When the biological activity of the biocarbon in the stored bio-carbon dioxide is transferred to the methane proportion reconverted to electric power with the fossil carbon, it is 850,000 kW of ecological power.

From 120 tons of wood and 80 tons of coal as well as 1 million kW of excess power, 300,000 kW of electric power and 130,000 cubic meters of methane, which is reconverted to 850,000 kW of ecological current, are obtained according to need and utilizing the variations in the power grid. Thus, all in all, about 1.1 million kW of ecological current is obtained. If the 120 t of wood was converted to electric power alone in the power plant, only about 250,000 kW of ecological current would be obtained, i.e., according to this process, the bioenergy yield achievable from biomass is increased by three to four times.

If the coal is predominant in the above approach, then the excess coal is converted to climate-friendly methane. Such methane, which consists of carbon of fossil origin and of hydrogen produced with ecological current, is referred to as “hybrid methane” in the following.

In detail, with the present technology:
about 1.1 million kW of ecological power (including the electric power generated in the first operational stage) is obtained from 120 tons of wood.
1 million kW of excess energy is stored in the gas network in the form of 130,000 cubic meters of methane.
these amounts of methane are reconverted to electricity with 85% efficiency, based on the originally employed electric power (cf. Annex: “electrochemical model calculation . . . ”).
the storage capacities for carbon dioxide are doubled with respect to their effect on the climate.
contributions to the reduction of carbon dioxide emission are increased four times by biomass as compared to being burned or gasified.
renewable energies are affordable by the carbon-dioxide-neutral use of cheap coal.

In practice, the synthesis gas obtained in the gasification of coal and wood contains a slight excess of hydrogen and preformed methane, so that the efficiency can be even higher in the reconversion to electricity of the originally employed excess electric power.

In this technology, it is important for a clean life cycle assessment to exactly adjust the biological fraction in the carbon dioxide sequestered in the first operational stage and in the methane produced in the second operational stage. However, this is difficult, for example, in seasonal variations in the coal-to-biomass ratio, in the demand-driven addition of natural gas to the synthesis gas in the first operational stage, in a varying ratio of the first to second operational stages, and in a varying offer of excess electric power (cf. Annex, Chapter “Survey of chemical reaction equations”).

One possibility is to determine C14, which is present only in the respective biological fraction, both in the sequestered carbon dioxide and in the methane supplied to the network according to the radiocarbon method (cf. in the Annex “Determination of the Biological Fraction in the Gases Carbon Dioxide and Methane”). The C14 can be determined quantitatively by modern methods, for example, by mass spectrometry.

The direct biological fraction of the two gases is determined by continuously measuring the carbon isotope C14 in the carbon dioxide sequestered in the first operational stage and in the methane produced in the second operational stage. Now, the bioactivity of the sequestered bio-carbon dioxide is transferred to fossil methane.

Since methane and carbon dioxide as chemical compounds both contain one carbon atom each and both are gases, the bioactivity can be transferred at a ratio of 1:1. The specific chemistry of carbon creates the preconditions for the multiplication of bioenergy according to the invention.

Thus, the oxygen formed in the electrolysis of water in the amount suitable for the combustion of the synthesis gas can be employed with advantage in the combustion of the synthesis gas or of the hydrogen in the power plant instead of combustion air (Reactions 3 and 4). Thus, in the absence of atmospheric nitrogen, the formation of nitrogen oxide, which is harmful to the climate, is excluded. The higher combustion temperature, which is due to the high energy density of oxygen/fuel mixtures, can be controlled by the addition of steam or carbon dioxide available on-site.

When hydrogen is burnt with oxygen, only water vapor is formed, which may condense and can be recirculated to electrolysis. When the synthesis gas is burnt with oxygen, only carbon dioxide remains after the water vapor has condensed, and the carbon dioxide can be directly sequestered.

If the conversion to electricity of the synthesis gas is combined with the electrolysis of water in a plant in the process according to the invention, and if these processes occur in successive operational stages, the connection to the high voltage grid and the transformers of the power plant can be utilized in both directions by both the power plant and the electrolysis of water. Also, if needed, the gas network could supply natural gas/methane to the gas power plant in the first operational stage, and then take up methane in the second operational stage.

Another advantage of this combination of plants is the fact that aqueous condensate formed in the combustion of hydrogen and methane can be separated off, stored and processed for the electrolysis of water. Similarly, the oxygen formed in the electrolysis of water can be stored and employed with advantage in gas combustion instead of air.

A central subject matter of the present invention is the multiplication of the bioenergy achievable from biomass, characterized in that biomass is chemically and/or thermally converted to bio-carbon dioxide and bio-hydrogen, the bio-carbon dioxide is stored/sequestered, and the bio-hydrogen is methanized with one of the carbon oxides, and that emission rights or bioactivity associated with the sequestering of the bio-carbon dioxide are determined by measurement and proportionally transferred to fossil carbon.

The offsetting of the emission rights can also be done within the plant by processing mixtures of fossil fuels (e.g., coal) and biomass (e.g., wood) according to the invention and transferring the emission rights gained by the proportion of bio-carbon in the stored carbon dioxide to the fossil carbon in the methane produced. In order to utilize the full potential of the bioactivity associated with the stored bio-carbon dioxide, the proportion of fossil carbon in the processed mixture should be more than half.

The bioenergy can then be further multiplied by employing additional hydrogen from the electrolysis of water from excess electric power according to the invention. This technology also yields a storage power plant with high efficiency in which electric power is supplied or taken up in successive operational stages, and can be stored and transported and reconverted to electricity in the form of methane. This is considered quantitatively in the last Chapter “Chemical compounds/mass/volume/energy”.

As compared to the combustion or gasification of biomass (biogas), the present process offers a multiple increase of the recovery of bioenergy.

Bioenergy allies itself with wind and solar power.

Climate objectives can be achieved more quickly.

Annex (The Annex is part of the description)

Description of the starting materials (biomass)

Decomposition of the biomass into hydrogen and carbon dioxide

Separating, introducing and processing the gases

Survey of the chemical reactions

Survey of the individual plants

Storage facilities and storage media

Synthesis gas, production and use

Synthesis gas/conversion to electricity/storage of carbon dioxide

Detection of the bio fraction in the gases carbon dioxide and methane

Electrochemical model calculation for the production of methane

Chemical compounds/mass/volume/energy

Description of the Starting Materials:

All variants of biomass may serve as the starting materials according to the invention. Preferably, these are plants that convert carbon dioxide to organic carbon compounds and oxygen by means of chlorophyll. These plants may grow on the land, in the waters, and in the sea. Plants are preferred because they contain little nitrogen, phosphorus and sulfur, in contrast to zoological biomass.

These basic materials may also be refined for use according to the invention. Thus, for example, ears may be threshed, and the cereals and straw may be processed separately. The same applies to maize. The refining may go even further, and the oil may be pressed from oil seeds and used separately. Or the by products/waste products of oil production are used according to the invention.

The biochemical refining products of biomass, such as biogas and bioethanol, deserve consideration. It is true that both can be simply reacted as gases in the reformer to hydrogen and CO₂, and the CO₂ formed can be sequestered. However, part of the CO₂ has already been formed and released into the atmosphere during the production thereof from biomass. With biogas, it is also possible to separate off methane, feed it into the gas network, and then use the same amount of natural gas according to the invention.

The use of whole plants or plant parts, which are then further processed in a comminuted form, is particularly economical. There may also be mentioned: Agricultural and silvicultural waste materials. In general, organic products of the waste industry may be included.

With many materials according to the invention, the economical efficiency of the process can be improved by the inclusion of high-energy fossil fuels. Seasonal supply bottlenecks, for example, with annual plants, can also be equalized by such additions. Thus, for example, it may be advantageous to improve the hydrogen yield by adding coal when municipal green waste is used. This is ecologically safe because CO₂ emission is excluded in the processes described. Under favorable conditions, the use of coal together with biomass in this process is more economical than the separate combustion of coal with the technically complicated and thermodynamically inefficient subsequent separation of the CO₂ from the flue gases and the subsequent sequestering thereof. In the process according to the invention, the CO₂ can be directly sequestered after the separation of hydrogen. In the coprocessing with coal, wood and wood-like materials are preferred.

Decomposition of the biomass into hydrogen and carbon dioxide:

All known chemical processes in which biomass reacts to hydrogen and CO₂ using heat and pressure with the addition of steam are preferred. If the starting materials are in the form of liquids or gases, a steam reformer can be used for the purpose. Solid materials are reacted in a fluidized bed process as with coal gasification.

The process is two-stage, as shown for the model methane (CH₄, biogas): In the first stage, methane reacts with 1 mole of water to 3 moles of hydrogen (H₂) and one mole of carbon monoxide (CO). In the second stage, CO reacts with water to CO₂ and H₂. Thus, 4 moles of H₂ and 1 mole of CO₂ is formed per mole of CH₄. Similarly, the reaction equations for other classes of biochemical compounds can be developed. With them, the reactions also take a two-stage course. If carbon is reacted with steam, a mixture of carbon monoxide and hydrogen is formed in the first stage. This mixture is called synthesis gas. The Chapters “Synthesis gas, production and use” and “Synthesis gas/conversion to electricity/storage of carbon dioxide” predominantly relate to synthesis gas from coal, but also essentially apply to synthesis gas from coal/wood mixtures.

When non-pretreated biomass, such as wood, or whole plants are used, a solid residue that is a suitable fertilizer in agriculture is formed in addition to the gases.

Separating, Introducing and Processing the Gases:

At first, gases containing sulfur and nitrogen, if any, should be separated off. Then, hydrogen and carbon dioxide are separated by technically established processes, for example, by utilizing the different boiling points. Now, the hydrogen can be supplied to the power and heat production, and the CO₂ can be sequestered. However, only hydrogen may be separated, and all the remaining gases may be sequestered.

According to the invention, it is also possible to introduce the separated hydrogen into a natural gas deposit, and to extract/displace the natural gas. For this purpose, it is appropriate to introduce the hydrogen, which is lightweight, into the top part of the deposit, and to withdraw the natural gas from the lower part. As mentioned above, because of the great physical and combustion-technological differences, it is appropriate to keep the two gases separated, if possible, over an extended time of extracting/displacing.

However, it may also be appropriate to introduce the hydrogen in such a way that mixing of the gases occurs. Also, the hydrogen may be introduced into a deposit while natural gas is still being extracted, for example, in order to maintain a desired extraction pressure in the deposit. If required, the hydrogen may also be directly fed into the gas network or into a particular natural gas line.

Then, if gas mixtures are present during the extraction and transport, they are varying in quality, because the hydrogen does not uniformly distribute in the deposit and in the pipe system, and therefore, a fluctuating gas mixture is extracted. In this gas mixture, either the hydrogen can be separated off according to usual processes and recirculated into a deposit for further displacement, or the gas mixture is standardized by adding hydrogen or natural gas later according to need. As a third possibility, the fluctuating gas mixture can be supplied to the consumer, wherein the hydrogen content/calorific value must be determined at the site of consumption, and the gas dosage (and the determination of the value) must be adapted to the calorific value. Because of the high expenditure in equipment, it is recommendable to use fluctuating gas mixtures preferably at sites of large consumption, such as in gas power plants, and preferably to feed it there, not in the gas network, but in selected pipelines. When the deposit is charged with hydrogen, the sequestering of CO₂ can begin.

The following Chapters, especially those that relate to reactions and plants, predominantly describe the recovery of methane with fossil carbon (hybrid methane), but similarly apply to the recovery of biomethane:

Survey of the Chemical Reaction Equations (Reaction 1. to Reaction 5.)

| | |
|---|---|
| Reaction 1.) | C + H₂O → CO + H₂ |
| Reaction 2.) | (CO + H₂) + 2H₂ → CH₄ + H₂O |
| Reaction 3.) | 2H₂O → 2H₂ + O₂ |
| Reaction 4.) | CO + H₂ + O₂ → CO₂ + H₂O |
| Reaction 5.) | CH₄ + 2O₂ → CO₂ + 2H₂O |
| Reaction 6.) | CO₂ + 4H₂ → CH₄ + 2H₂O |

Survey of the Individual Plants of the Hybrid Storage Power Plant (In Parentheses: the Above Reactions Reaction 1. to Reaction 5. That Belong to the Respective Plant)

1. Power plant/gas power plant (Reaction 4. and/or Reaction 5.)

2. Plant for coal gasification and production of the synthesis gas (Reaction 1.)

3. Electrolysis device and rectifier for conversion of electric power to hydrogen (Reaction 3.)

4. Plant for the hydrogenation of carbon monoxide (or carbon dioxide) to hybrid methane (Reactions 5. and 6.)

5. Connection to the high voltage grid and transformer (Reactions 4./5. or Reaction 3.)

6. Connection to the natural gas network (Reaction 5. or Reaction 2.)

Storage Facilities and Storage Media

The most important storage facility is the gas network with hybrid methane as the storage medium. When needed, the stored hybrid methane or its equivalent of natural gas present in the gas network can then be reconverted to electricity. This reconversion to electricity is preferably effected in a gas power plant assigned to the hybrid storage power plant. The synergies occurring in this combination of plants are described in some detail above. However, the reconversion to electricity may also be effected in a more distant place, where the hybrid methane or its equivalents of natural gas are then withdrawn from the gas network.

The carbon dioxide may also be separated from the flue gases and stored or sequestered. If oxygen from the electrolysis of water is employed instead of air in the combustion, the carbon dioxide will remain as a gas after the condensation of water. If the carbon dioxide is also liquefied under pressure, carbon monoxide, which is unavoidable in coal combustion, remains and can be recirculated into the burner, so that it is not released into the environment.

Another storage medium is the feeding water for electrolysis, which is obtained as condensation water from the flue gases of the gas power plant or plants. If the gas power plant is connected with the hybrid storage power plant, the feeding water can be collected on-site, processed and stored in the tank with a corresponding capacity. From more remote gas power plants, the condensation water collected there would have to be transported to the hybrid storage power plant in tank trucks. In this case, condensates from condensing boilers could also be included in these transports. The invention relates to the collection and storage of the condensate from the natural gas/hybrid gas combustion, because the recovery of hybrid methane from synthesis gas is enabled as the quantity increases (Reactions 2., 3., and 5.). Because of its higher purity, the condensate from the combustion of natural gas is to be preferred over the condensate from the combustion of synthesis gas derived from coal, to be used for the electrolysis of water according to the invention.

Synthesis Gas/Production and Use

In the first stage of the “Fischer-Tropsch” process, the synthesis gas is formed from carbon and water vapor at high temperatures (Reaction 1.). Depending on the quality of the coal or the carbon compound, it contains carbon monoxide and hydrogen as a main component, and possibly methane. It is also possible to heat the coal with exclusion of air to 1000° C. to 1300° C. to obtain coke, i.e., purer carbon, which is reacted to synthesis gas. In addition, per about one ton of coal, there is obtained about 300 cubic meters of coal gas, a gas mixture with about 50% hydrogen and 30% methane as main components, which can be directly fed into the gas network or into Reaction 2. As another by-product of the coking of coal, the so-called “coal tar” is obtained, a mixture of aromatics. Historically, coal tar has been the starting point of the chemical industry. If the ecological ban is taken from coal with the process according to the invention, many chemical intermediates can again be recovered in the coal utilization according to the invention, and the dependency of chemistry on petrochemistry is reduced.

In both cases, the production of the synthesis gas, which includes its purification, is a complex continuously proceeding process in which constantly repeated starting and stopping in the changing operational stages of the storage power plant is prohibited. Therefore, it is a particular subject matter of the present invention that the synthesis gas is employed in different uses in both operational stages (in the first operational stage: according to Reaction 3., and in the second operational stage: according to Reaction 4.).

If the hybrid storage power plant is provided close to a coal power plant, the synthesis gas can also be blown into the combustion site of the coal power plant in the second operational stage, and thus converted to electricity. With an additional gaseous fuel, a higher power for peaks in demand is available essentially more quickly. Thus, flexibility is gained even with a coal power plant.

The conversion of the synthesis gas to hybrid methane (Reaction 2.) is effected in a reaction named after the chemist “Sabatier”, in which carbon monoxide is hydrogenated with hydrogen to methane on nickel or iron catalysts. The chemical reaction is exothermic and can be utilized thermally when the process according to the invention is refined, whereby the efficiency of the reconversion to electricity can be enhanced further.

When the reaction control in Reaction 3 is changed, long-chained hydrocarbons, which are suitable as fuels for motor vehicles, may also be obtained.

Synthesis Gas/Conversion to Electricity/Storage of Carbon Dioxide

“Conversion to electricity” of the synthesis gas means its direct or indirect thermal utilization for the purpose of producing electric power.

The carbon dioxide formed in the operational stage of conversion to electricity of the synthesis gas can also be stored/sequestered. For example, after the condensation of the water formed from the hydrogen during combustion, the carbon dioxide is separated from the flue gases by liquefaction under pressure. If the oxygen formed in the electrolysis of water is used for combustion instead of air, carbon dioxide is the only gas that remains after the condensation of water, which can be stored directly.

In the conversion to electricity of the synthesis gas, in addition to directly burning it, it is also possible to convert the carbon monoxide with steam to carbon dioxide and further hydrogen. Then, the carbon dioxide is stored, and subsequently, hydrogen is burnt exclusively. This hydrogen may also be methanized in the same way as hydrogen obtained from electrolysis. This is effected by reacting hydrogen with either stored carbon dioxide (Reaction 6.) or with synthesis gas/carbon monoxide (Reaction 2.). To the latter, the synthesis gas can be divided, wherein part thereof reacts to completion as above to hydrogen and carbon dioxide, and the remaining part of the synthesis gas then reacts with hydrogen to methane (Reaction 2.). Methane is also formed in the operational stage of conversion to electricity of the synthesis gas, which methane can also be stored alternatively for direct combustion/conversion to electricity.

To conclude, the synthesis gas can be converted to electricity/burnt as such, as hydrogen, or as methane. In all three variants, the carbon dioxide can be separated off and stored as described above.

The conversion of the synthesis gas to methane, even in the operational stage in which it should otherwise be converted to electricity, is recommendable if electric power is not needed in the place of the hybrid storage power plant, and cannot be conducted away either.

If the synthesis gas is obtained from biomass (e.g., wood) in the process according to the invention, the carbon dioxide that the plants withdrew from the atmosphere is stored in the soil in the sequestering in the operational stage of conversion to electricity, and biomethane is produced in the operational stage of storing excess energy.

Detection of the Bio Fraction in the Gases Carbon Dioxide and Methane

The gases carbon dioxide and methane formed as the end phase are either charged with fees or financially supported (e.g., biomethane), depending on their origin (biological or fossil). Therefore, it is important to determine the bio fraction in the above mentioned gases if, for example, varying proportions of wood are gasified with coal according to the invention.

This can be done by the radiocarbon method (C14 method) known from archaeology. In this method, it is considered that the biomass employed and thus the produced biomethane have the initial value with respect to the C14 isotope proportion, while fossil carbon contains no C14. The same applies to carbon dioxide. The measurement can be effected on the gases according to the so-called “Libby counter tube method”.

Electrochemical Model Calculation for the Production of Hybrid Methane From (Excess) Electric Power and Coal:

Starting with Reaction 3 (electrolysis of water), 4.2 kW is required for one cubic meter of hydrogen (H₂) for an assumed efficiency of the electrolysis of 80%. According to Reaction 2, another 2 moles of hydrogen (H₂) is required for the production of hybrid methane from carbon monoxide, in addition to the hydrogen of the synthesis gas. Therefrom, it follows that about 8.4 kW of electric power is required per cubic meter of hybrid methane (CH₄) produced from synthesis gas.

It is assumed that the carbon for the hybrid methane is obtained from coal. Methane consists of 75% carbon (molecular weight of methane: 16, atomic weight of carbon: 12). The gas density of methane is at 718 g/cubic meters. It can be calculated therefrom that 1 cubic meter of methane contains 539 g of carbon. With a carbon content of coal of from 65% to 90% (depending on the quality of the coal), from 580 g to 830 g of coal is required per cubic meter of hybrid methane.

In summary, 8.4 kW of (excess) electric power and from 580 g to 830 g of (dry) coal yield one cubic meter of hybrid methane, which is comparable with natural gas of H quality. Reconverted to electricity, a cubic meter of hybrid methane would yield 7.5 kW (energy content of hybrid methane: 11.5 kW/efficiency of the gas power plant: 65%). If the use of the coal (580 g) is left unconsidered, the efficiency of the reconversion to electricity is 87%.

Chemical Compounds/Mass/Volume//Energy

| | | |
|---|---|---|
| Reactions | Reaction 1 | C + H₂O → (CO + H₂) |
| | Reaction 2 | (CO + H₂) + 2H₂ → CH₄ + H₂O |
| | Reaction 3 | 2H₂O → O₂ + 2H₂ |
| | Reaction 4 | (CO + H₂) + O₂ → CO₂ + H₂O |
| | Reaction 5 | CH₄ + 2O₂ → CO₂ + 2H₂O |
| | Reaction 6 | CO₂ + 4H₂ → CH₄ + 2H₂O |
| | Reaction 7 | CO + H₂O → CO₂ + H₂ |

1. Approach:

60 t of bio-carbon (=120 t of wood)+60 t of fossil carbon (=80 t of coal)

In the above mentioned reactions, 60 tons of carbon yields about 120,000 cubic meters of CO₂, CO or CH₄ gas. Multiples for H₂, O₂ and H₂O vapor then result from the respective reactions. (All numerical values are coarsely rounded/note: (CO+H₂) in Reactions 1, 2 and 4 is synthesis gas.)

Doubling of Bioenergy:

The successive reactions Reaction 1 and Reaction 7 yield 240,000 cubic meters of CO₂ (equal shares of biological and fossil CO₂) and 480,000 cubic meters of H₂. With half of the CO₂ according to Reaction 6, H₂ yields 120,000 cubic meters of CH₄.

| Balance: | sequestered CO₂ | methane produced: |
|---|---|---|
| | 60,000 cbm biol. CO₂ -bioactivity→ | 60,000 cbm fossil CH₄ |
| | 60,000 cbm fossil CO₂ | 60,000 cbm biol. CH₄ |
| | | 120,000 cbm “biomethane” |

The same balance is obtained if, after Reaction 1, the synthesis gas is divided, half of the CO in the synthesis gas reacts to CO₂ and additional H₂ according to Reaction 7, CO₂ is sequestered, and 2H₂ reacts with the other half of the synthesis gas to CH₄ and H₂O according to Reaction 2.

120,000 cubic meters of methane/natural gas, converted to electricity in a G+D power plant, yields about 800,000 kW of ecological power.

Threefold to Fourfold Bioenergy:

In a storage power plant, half of the synthesis gas is converted to electricity in a gas power plant in the first operational stage to obtain about 400,000 kW in a CO₂-free manner. The CO₂, which consists of equal shares of biological and fossil CO₂ (as above 120,000 cbm), is sequestered.

The other half of the synthesis gas is reacted with 240,000 cubic meters of H₂, which is obtained in the second operational stage by the electrolysis of water from 1 million kW of excess electric power according to Reaction 3, to yield 120,000 cubic meters of methane according to Reaction 2. The balance and transfer of the bioactivity is as above. Here too, about 800,000 kW of ecological power is obtained. Together with the 400,000 kW from the first operational stage, 1.2 million kW of (CO₂-neutral) ecological power is obtained according to this variant.

120 tons of wood, converted to electricity separately in a power plant, yields only 300,000 to 400,000 kW of ecological power.

One feature of the present invention is the fact that the quantities in the chemical reactions are exactly matching. Therefore, the quantitative ratios in the biological and fossil raw materials should be selected so that fossil methane, to which the bioactivity of stored bio-carbon dioxide can be transferred, is always sufficiently produced.

1-15. (canceled)

16. A process for the transport and steadying of fluctuating wind and solar power in the natural gas network by obtaining hydrogen from electric power by the electrolysis of water, reacting the hydrogen with synthesis gas to methane, and feeding the methane produced into the natural gas network, and reconverting to electricity the thus stored and transported methane or its natural gas equivalent in a different place, wherein the hydrogen from the electrolysis of water converts the entire synthesis gas to methane.

17. The process according to claim 16, in which the synthesis gas is formed from carbon and steam in accordance with the first stage of the “Fischer-Tropsch” process.

18. The process according to claim 16, in which the synthesis gas is formed with coal.

19. The process according to claim 16, in which the synthesis gas is formed with biomass, preferably from plants.

20. The process according to claim 16, in which the synthesis gas is formed with sugar.

21. The process according to claim 16, in which the synthesis gas is formed with methane.

22. The process according to claim 16, in which the carbon dioxide formed in the flue gases during the conversion to electricity of the methane or natural gas is separated off and stored/sequestered.

23. The process according to claim 16, in which, when mixtures of biological and fossil carbon are used for producing the synthesis gas, the biological proportion in the produced methane or in the stored/sequestered carbon dioxide is determined by the C14 (radiocarbon) method.

24. The process according to claim 16, in which the water vapor contained in the flue gases in the conversion to electricity of methane/natural gas is condensed and used as feeding water in the electrolysis of water.

25. The process according to claim 16, in which the oxygen formed in the electrolysis is collected and stored, and employed in the combustion of methane instead of combustion air.

26. The process according to claim 16, in which methane is decomposed to hydrogen and carbon dioxide, and the hydrogen is converted to electricity, and in the decomposition, 1 mole of methane yields 4 moles of hydrogen and one mole of carbon dioxide.

27. The process according to claim 22, in which stored carbon dioxide is reacted with hydrogen at a molar ratio of 1:4 to form methane.

28. The process according to claim 16, in which the methane produced from wind and solar power is transported in gas pipes.
